(19) United States
(12) Patent Application Publication
(10) Pub. No.: US 2011/0313322 A1
(43) Pub. Date: Dec. 22, 2011

(54) LIQUID PRESSURE FORCE SENSOR

(76) Inventor: Helge Fossan, Stavanger (NO)

(21) Appl. No.: 13/091,245

(22) Filed: Apr. 21, 2011

(30) Foreign Application Priority Data
May 6, 2010 (NO) 2010 0654

(51) Int. Cl. A61B 5/103

(52) U.S. Cl. 600/587

(57) ABSTRACT

The invention provides a force sensor device for measuring force when applying compressions or simulating applying compressions to a chest area of a person during cardiopulmonary resuscitation (CPR). More specifically, the device has the shape of a flat pad or similar with a top side and a bottom side, for being placed on a chest area of a man or manikin or on any feasible surface or object, preferably with the top side of the device facing up. The device comprises a volume that is filled with a liquid, the device is sufficiently high and rigid to maintain a distance between the top and bottom sides of the device when normal CPR compressions are applied on the device, and the device comprises at least one pressure sensor arranged with fluid contact with the liquid volume and in operative contact with means for force measurement as a function of liquid pressure that varies according to applied pressure during CPR.

### FIELD OF THE INVENTION

The present invention relates to health and life saving. More specifically, the invention relates to a force sensor device for measuring force when applying compressions or simulating applying compressions to a chest area of a person during cardiopulmonary resuscitation (CPR).

### BACKGROUND OF THE INVENTION AND PRIOR ART

When giving CPR for life saving, correct level and rate of applied force to the chest is crucial for having a good success rate. Realistic training is crucial in that respect.

Force sensor devices useful for measuring force when applying compressions for CPR already exist. The existing devices are based on strain measurements, using strain gauges, or use compressible conductive tape. However, they are rather expensive and complicated, they could be even more reliable and they are susceptible to hysteresis effects, temperature drift and creep. Some related art in this respect can be found in the patent publications U.S. Pat. No. 4,166,458A, US 2009/255344 A1, EP 1057451 A2, U.S. Pat. No. 3,652,925 A, U.S. Pat. No. 5,589,639 A, GB 1526021 A and U.S. Pat. No. 4,019,501 A. A demand therefore exists for an alternative force sensor device that is beneficial compared to the existing devices, and the objective of the present invention is to meet the demand.

### SUMMARY OF THE INVENTION

The objective of the invention is met by providing a force sensor device for measuring force when applying compressions or simulating applying compressions to a chest area of a person during cardiopulmonary resuscitation (CPR). More specifically, the device has the shape of a flat pad or similar with a top side and a bottom side, for being placed on a chest area of a man or manikin or on any feasible surface or object, preferably with the top side of the device facing up. The device comprises a volume that is filled with a liquid, the device is sufficiently high and rigid to maintain a distance between the top and bottom sides of the device when normal CPR compressions are applied on the device, and the device comprises at least one pressure sensor arranged with fluid contact with the liquid volume and in operative contact with means for force measurement as a function of liquid pressure that varies according to applied pressure during CPR.

Accordingly, the device is a unit to be placed between the hand(s) applying compressive force and an underlayer, be it the chest of a person, a manikin or any feasible underlayer. The device can be rather soft or rigid, but it contains a liquid filled volume and a pressure sensor in operative contact in order to sense the liquid pressure of said volume, and the design of the device is so that the volume is not pressed flat when the device is subject to normal forces for CPR, preferably not even when applying hard CPR compressions. The pressure varies linearly or non-linearly with applied force, depending on the design of the device and the extent of liquid filling of the volume, however, the pressure and force is operatively correlated in order to provide correct measurements of force applied on the device.

Preferably the device comprises a stiff top plate on the top side and a distance ring that is arranged around the sides of the device, the top plate is elastically arranged to the distance ring, within the inner periphery of the distance ring.

Preferably the device has an oval disc shape, the top side comprises a stiff top plate and an elastic top layer connected to the top plate over or under the top plate, the top plate is slightly smaller than the elastic top layer that extends out to and is fastened to an oval distance ring, whereby the top plate is elastically connected to the distance ring, the distance ring is arranged around the oval periphery of the device, the bottom side comprises a printed circuit board (PCB) having arranged to it a pressure sensor in fluid contact with the liquid volume and the PCB comprises or is in operative contact with means for force measurement as a function of liquid pressure that varies according to applied pressure during CPR.

Preferably the distance ring is rigid, oval and larger than the stiff pressure plate on the top side of the device, the top plate is arranged at higher level than a top side of the distance ring to which the top plate is elastically connected with an elastic connection that obliquely extends upward from the top side of the distance ring to the outer periphery of the top plate, such that applying typical CPR compression force to the top plate will result in some bulging of the elastic connection but preferably not to such extent that the top of the bulge extends to a level above the top side of the device. Preferably the device is designed so that the bulge, when applying a typical maximum force for CPR compressions, extends up to the same level as the top side, whereby an in substance linear relation between force and liquid pressure is achieved. Preferably the elastic connection or suspension of the top plate is in substance in-plane with the plate during compression, whereby the top plate that is to receive the applied compressions and the liquid pressure correlates in substance directly to the pressure applied on the top plate, since the forces in-plane with the plate are perpendicular and therefore will not contribute.

Preferably the force sensor device has an opening for filling of liquid, and the opening can be plugged after filling. The liquid is any in substance incompressible liquid, preferably an inert liquid such as an inert mineral oil or synthetic oil, for example so called baby oil.

The force sensor device according to the invention preferably comprises a PCB with electronic means for determining and transmitting values of applied force or a signal corresponding to values of applied force to a display integrated in the force sensor device, the display providing real time display of applied force and in addition information for guiding in order to correct incorrect values of force and guiding in order to correct incorrect rate of compressions. The guiding may also comprise audible signals.

The force sensor device comprises at least one, preferably at least two, pressure sensors chosen amongst any convenient liquid pressure sensors, for example monocrystalline silicon pressure sensors, piezoelectric pressure sensors and piezoresistive pressure sensors. The device preferably comprises at least two pressure sensors, at least one of which is exposed to ambient temperature and pressure in order to compensate for any drift due to variations in ambient temperature and pressure.

### FIGURES

The invention is illustrated with figures, of which

FIG. 1 illustrates an embodiment of a force sensor device according to the invention, in longitudinal cross section and as viewed from the top side,

FIG. 2 illustrates the components of the force sensor device of FIG. 1,

FIG. 3 illustrates details of the elastic connection between a rigid top plate and a side element, and

FIG. 4 illustrate a complete force sensor device according to the invention, including a display.

### DETAILED DESCRIPTION

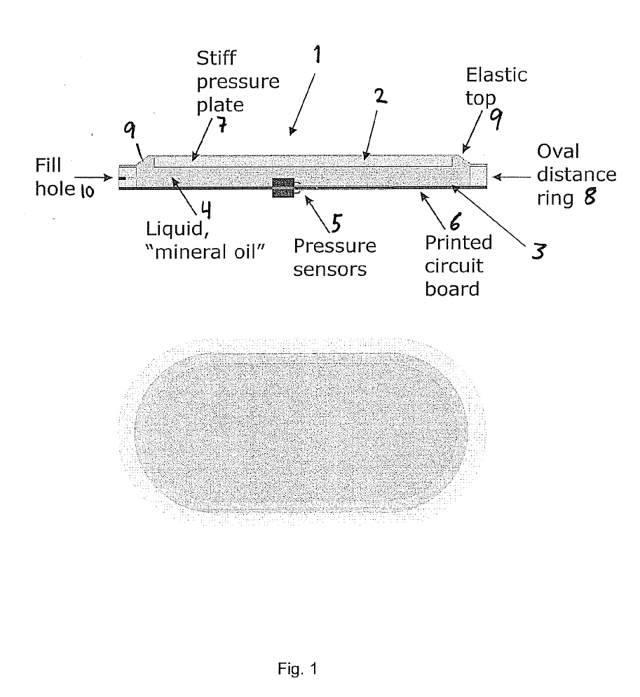

Reference is first made to FIG. 1, illustrating an embodiment of a force sensor device according to the invention, in longitudinal cross section and as viewed from the top side. More specifically, it is illustrated that the device 1 has the shape of a flat pad or similar with a top side 2 and a bottom side 3, for being placed on a chest area of a man or manikin or on any feasible surface or object, the device comprises a volume 4 that is filled with a liquid, the device is sufficiently high and rigid to maintain a distance between the top and bottom sides of the device when normal CPR compressions are applied on the device, and the device comprises at least one pressure sensor 5 arranged with fluid contact with the liquid volume and in operative contact 6 with means for force measurement as a function of liquid pressure that varies according to applied pressure during CPR.

The illustrated embodiment comprises a stiff or rigid top plate 7, an oval distance ring 8 constituting the sides of the device, an elastic connection between 9 the top plate and a top side of the distance ring, a printed circuit board (PCB) constituting the bottom side, two pressure sensors 5, a liquid fill hole 10 in the distance ring and a mineral oil filling the volume 4 of the device. The pressure sensors, one of which senses the liquid pressure and one senses the ambient pressure, are cabled to the PCB, which provides means for converting the sensed pressure signals to force or a representative parameter for force.

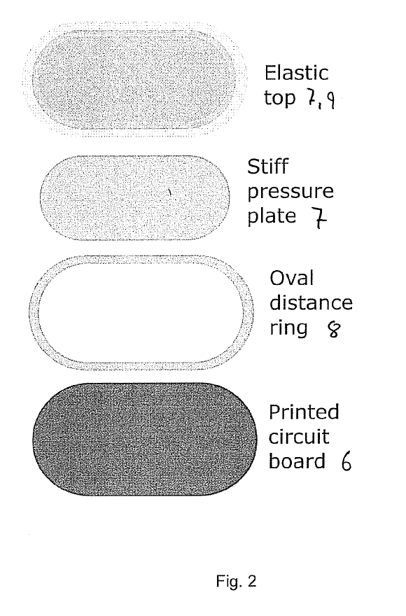

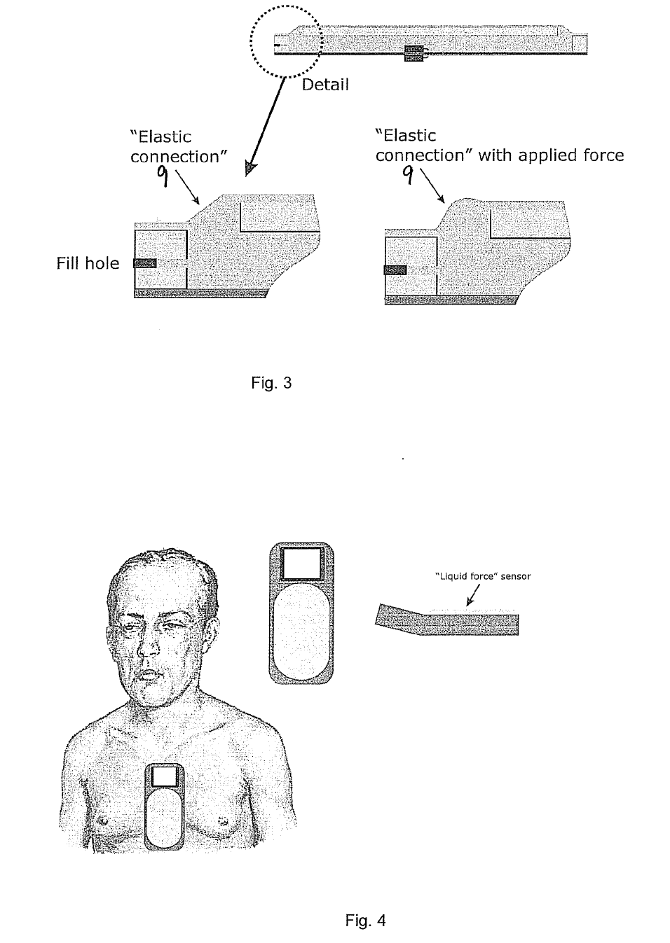

FIG. 2 illustrates the sensor device of FIG. 1 with the components disassembled. Further details are evident from FIG. 3, illustrating the elastic connection, which can have many different embodiments. Preferably the elastic connection or suspension of the top plate is in substance in-plane with the plate during compression, since the forces in-plane with the plate will not contribute to the resulting liquid pressure. More specifically, the applied compression force and the liquid pressure act in substance perpendicular to the top plate, and the elastic connection is perpendicular to said force and pressure, thereby not affecting the correlation between applied force and resulting liquid pressure. If the elastic connection, and any further deformation of the device, has a spring-like linear relation to the applied force, the correlation between applied force and liquid pressure is still in substance linear.

The elastic connection will make the device rather insensitive to temperature variations, as said connection acts as a pressure compensator. The differential pressure between the two sensors will give an accurate reading of the liquid pressure and applied force. Contrary to prior art force sensors, the present design is in substance unaffected by hysteresis and creep. The actual pressure sensors are not subject to deformation, contrary to prior art force sensors. The components of the device can for example be injection moulded in one unit in one operation, to a large extent. The top side, the sides and elastic connection between them can conveniently be injection moulded as one unit in a two component moulding process.

FIG. 4 illustrates how the force sensor device is included in or acts as a CPR sensor device, which CPR device preferably comprises means for providing guidance as to level or extent of applied force, the rate of applied force, and preferably also further parameters that are important for proper training or practice of CPR.

Many different shapes and design will be possible within the scope of the invention, including any operative combination of features as described or illustrated. Reliability and cost may for some embodiments be governing for the design, whilst realism and accuracy may be governing for other embodiments.

1. A force sensor device, comprising:
a flat pad comprising a top side and a bottom side;
a volume filled with liquid in the flat pad;
a stiffness in the top side and sides of the flat pad;
at least one pressure sensor arranged in the volume or with fluid contact with the volume filled with the liquid;
means for force measurement as a function of liquid pressure as measured by said at least one pressure sensor;
wherein, when applying compressions or simulating applying compressions to a chest area of a person during cardiopulmonary resuscitation (CPR) and having the force sensor device on a chest area of the person or manikin or on any feasible object for receiving the compressions, the applied pressure during CPR can be measured.

2. The force sensor device according to claim 1, wherein the force sensor device is sufficiently high and rigid to maintain a distance between the top and bottom sides of the force sensor device when normal CPR compressions are applied on the force sensor device.

3. The force sensor device according to claim 1 further comprising:
a stiff top plate on the top side;
a distance ring arranged around sides of the force sensor device;
wherein the stiff top plate is elastically arranged to the distance ring within an inner periphery of the distance ring which is sufficiently high and rigid to maintain a distance between the top and bottom sides of the force sensor device even when hard CPR compressions are applied on the top side.

4. The force sensor device according to claim 1, wherein the force sensor device comprises two pressure sensors, wherein at least one of the two pressure sensors is exposed to ambient pressure and temperature.

5. The force sensor device according to claim 1, wherein:
the force sensor device comprises an oval disc shape;
the top side comprises a stiff top plate and an elastic top layer connected to the stiff top plate over or under the stiff top plate;
the stiff top plate being slightly smaller than the elastic top layer that extends out to and is fastened to an oval distance ring;
the stiff top plate being elastically connected to the oval distance ring, the oval distance ring being arranged around an oval periphery of the force sensor device;
the bottom side comprises a printed circuit board (PCB) comprising the at least one pressure sensor in fluid contact with the liquid volume; and
the PCB comprises or is in operative contact with means for force measurement as a function of liquid pressure that varies according to applied pressure during CPR.

6. The force sensor device according to claim 3, wherein the distance ring is rigid, oval and larger than the stiff top plate on the top side of the force sensor device;
wherein the stiff top plate is arranged at higher level than a top side of the distance ring to which the stiff top plate is elastically connected with an elastic connection that obliquely extends upward from the top side of the distance ring to an outer periphery of the stiff top plate; and
wherein application of a CPR compression force to the stiff top plate results in some bulging of the elastic connection but not to such extent that a top of a bulge extends to a level above the top side of the force sensor device.

7. The force sensor device according to claim 6, wherein the bulge, when applying a maximum force for CPR compressions, extends up to the same level as the top side, wherein an in substance linear relation between force and liquid pressure is achieved.

8. The force sensor device according to claim 1, wherein the force sensor device comprises an opening for filling the liquid, wherein the liquid comprises an inert mineral oil or synthetic oil, and wherein the opening is plugged after filling.

9. The force sensor device according to claim 1, wherein the force sensor device comprises a printed circuit board (PCB);
wherein the PCB comprises electronic means for determining and transmitting values of applied force or a signal corresponding to values of the applied force to a display integrated in the force sensor device; and
wherein the display provides at least one of real time display of the applied force and information for guiding in order to correct incorrect values of force and guiding in order to correct incorrect rate of compressions.

10. The force sensor device according to claim 1, wherein the force sensor device comprises at least one, preferably at least two, pressure sensors selected from the group consisting of monocrystalline silicon pressure sensors, piezoelectric pressure sensors and piezoresistive pressure sensors and combinations thereof.
